(19) United States
(12) Patent Application Publication
(10) Pub. No.: US 2023/0383225 A1
(43) Pub. Date: Nov. 30, 2023

(54) LUNG-ON-CHIP DEVICE WITH INTEGRATED EXTRACELLULAR MATRIX MEMBRANE

(71) Applicant: University of Utah Research Foundation, Salt Lake City, UT (US)

(72) Inventors: Matt D. Nelson, Sale Lake City, UT (US); Bruce K. Gale, Salt Lake City, UT (US)

(21) Appl. No.: 18/202,764

(22) Filed: May 26, 2023

Related U.S. Application Data
(60) Provisional application No. 63/346,595, filed on May 27, 2022.

(51) Int. Cl. C12M 3/00; C12M 3/06; C12M 1/12

(52) U.S. Cl. CPC C12M 21/08; C12M 23/16; C12M 25/14

(57) ABSTRACT

Disclosed are microphysiological systems (MPS) that may be used to model microenvironments of the human lung. The system includes a body, having one or more vacuum channels and a circulation channel separated by one or more flexible barriers. An aperture exposes the circulation channel to the top surface of the body, over which a culturing membrane, having epithelial and endothelial cells, may be placed. The cells of the culturing membrane may be subjected to radial strain and shear forces by causing a fluid to flow through the circulation channel and applying a vacuum to the one or more vacuum channels, causing the one or more flexible barriers and the culturing membrane to deflect.

### CROSS-REFERENCE TO RELATED APPLICATIONS

This application claims priority to and the benefit of U.S. Provisional Patent Application Ser. No. 63/346,595, filed May 27, 2022 and titled “Lung-On-Chip Device With Integrated Extracellular Matrix Membrane,” the entirety of which is incorporated herein by this reference.

### BACKGROUND

Currently, the lung is studied with a combination of conventional in-vitro cell culture and animal models, which are both limited in their ability to model human in-vivo cellular microenvironments. Cells are typically studied in-vitro as a single monolayer in conditions unlike their native environment, which includes other cell types, ECM interactions, and dynamic mechanical forces that all play a major role in their activity and differentiation. Permeable supports (i.e., cell culture inserts) have expanded in-vitro cell culture through the incorporation of a permeable membrane amenable to cell co-culture models, but these models are not capable of flow or cyclic strain, and the permeable membrane is composed of synthetic polymers. Animal models can provide complex information about pathology and drug response, but animals are costly and time-consuming to maintain, difficult to image, and exhibit significant differences in the expression, substrate specificity, tissue distribution, and abundance of drug transporters.

Microphysiological systems (MPS) emerged through the convergence of microfluidics and tissue engineering with the goal of creating sophisticated in-vitro models that more accurately recapitulate functional units of human organs. Soft lithography with polydimethylsiloxane (PDMS) enabled researchers to create dynamic lung microphysiological systems that were used to study nanoparticle inhalation, pulmonary edema, and intravascular thrombosis, among others. Other lung microphysiological systems have employed a microdiaphragm with breathing more similar to that of the native lung. While the importance of PDMS in early organ-on-a-chip models cannot be understated, the polymer has several limitations that will likely limit its future in the field—namely, its manual and, in some cases, highly technical fabrication, and limited architectures. This is a significant and unaddressed problem, as more complex architectures are often needed to recapitulate cellular microenvironments, and devices need to be scalable to be useful for preclinical trials. Accordingly, a need exists for an improved MPS that better models the lung microenvironment at reduced cost with less technical fabrication.

### SUMMARY

The present disclosure relates to lung microphysiological systems, comprising a body, a first microfluidic feature including one or more vacuum channels extending into the body from a vacuum inlet to a terminal portion within the body, and a second microfluidic feature comprising a circulation channel extending into the body from a circulation inlet and exiting the body at a circulation outlet, at least a portion of the circulation channel in proximity with a portion of the one or more vacuum channels, each of the one or more vacuum channels being separated from the circulation channel by a flexible barrier. The body and each flexible barrier may be integrally formed as a single, contiguous material. This may be achieved through, for example, SLA, MSLA, and DLP 3D printing methods.

An aperture may be formed that exposes the circulation channel to a top surface of the body. A culturing membrane may be disposed over the aperture, upon which epithelial and endothelial cells may be deposited. The culturing membrane may comprise an extracellular matrix (ECM) membrane, including proteins from the matrisome of the human lung, such as collagens and elastin.

The culturing membrane may be subjected to cyclic radial strain and shear forces. The culturing membrane may be exposed to cyclic radial strain by providing a vacuum to the one or more vacuum channels. Doing so causes the flexible barrier to deflect and in turn causes a pressure differential between the vacuum channel and the circulation channel. The pressure differential causes the culturing membrane to also deflect in a generally downward direction. Shear forces may be imparted to the culturing membrane by flowing cell media or other fluid through the circulation channel.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates a schematic of the distal lung.

FIG. 2 illustrates an exemplary lung MPS.

FIGS. 3A and 3B illustrate a cross-section view of the cell component of an exemplary lung MPS, in an unstretched and stretched configuration, respectively.

FIG. 4 illustrates an exemplary lung MPS for introducing cyclic radial strain to the cells of the system.

FIGS. 5A and 5B illustrate a front cross-sectional view of an exemplary MPS in a relaxed and flexed state, respectively.

FIG. 6 illustrates a top cross-sectional view of an exemplary MPS.

### DETAILED DESCRIPTION

### Alveolar-Capillary Barrier

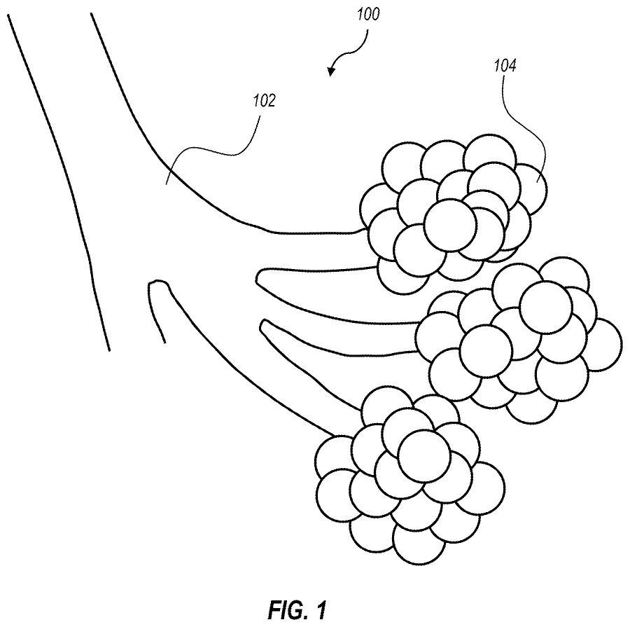

FIG. 1 illustrates an exemplary structure 100 of the terminal part of the human lung. The lung is composed of a branching structure dividing from bronchioles 102 into alveolar ducts, to alveolar sacs 104 containing alveoli. Oxygen is exchanged from the alveoli to the blood via the alveolar-capillary barrier. The barrier is thin (ranging from a thickness of approximately 200 nm at its narrowest to approximately 2 μm) and comprises an extracellular matrix bounded on one side by a layer of epithelial cells and on the other side by a layer of endothelial cells.

During respiration, the alveoli expand and contract, exchanging oxygen and carbon dioxide with the pulmonary capillaries. The alveolar epithelium is composed of alveolar type I (ATI) cells and alveolar type II (ATII) cells. ATI cells are non-proliferating cells responsible for gas exchange, which flatten and spread their cytoplasm to cover over 90% of the alveolar surface area to maximize diffusion. ATII cells are the progenitors for ATI cells and are responsible for the production of pulmonary surfactant. The two main mechanical forces experienced in the distal lung are cyclic strain (imparted from the expanding alveoli during respiration) and shear forces (as a result of capillary flow). The respiration rate of a healthy adult human is approximately 12 to approximately 20 breaths per minute, and the corresponding linear distension of the alveoli is estimated at 4% for a resting rate and 12% for a deep inspiration.

The pathophysiology of many global respiratory diseases, such as chronic obstructive pulmonary disease (COPD), lung cancer, and tuberculosis, involves the alveolar-capillary barrier. For example, emphysema is a component of COPD in which the alveoli rupture and gradually form one large pocket, reducing the surface area available for diffusion and therefore worsening breathing. These respiratory diseases result in millions of deaths every year and affect hundreds of millions of patients.

The development of pharmaceuticals for treating respiratory diseases has been slowed by the limitations of available models of the lung. In-vitro and animal studies have been limited in their ability to simulate the human lung in-vivo cellular microenvironment. The development of microphysiological systems (MPS) has proved an important aid to the development of therapies for treating damaged or infected lungs. Recent developments in microfluidics technology have enabled MPSs to provide relatively quick development of cell cultures at less cost.

### Overview of Microphysiological Systems

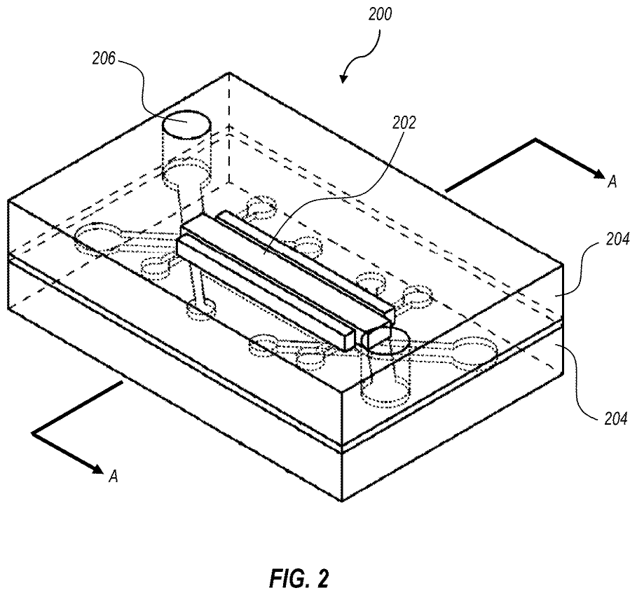

FIG. 2 illustrates an exemplary traditional MPS 200 for modeling the alveolus microenvironment. The system 200 includes a cell component 202 situated between two plates 204, the cell component 202 and the plates 204 including a plurality of microfluidic channels 206 for providing access to the cell component 202 for delivering media and actuating the system 200.

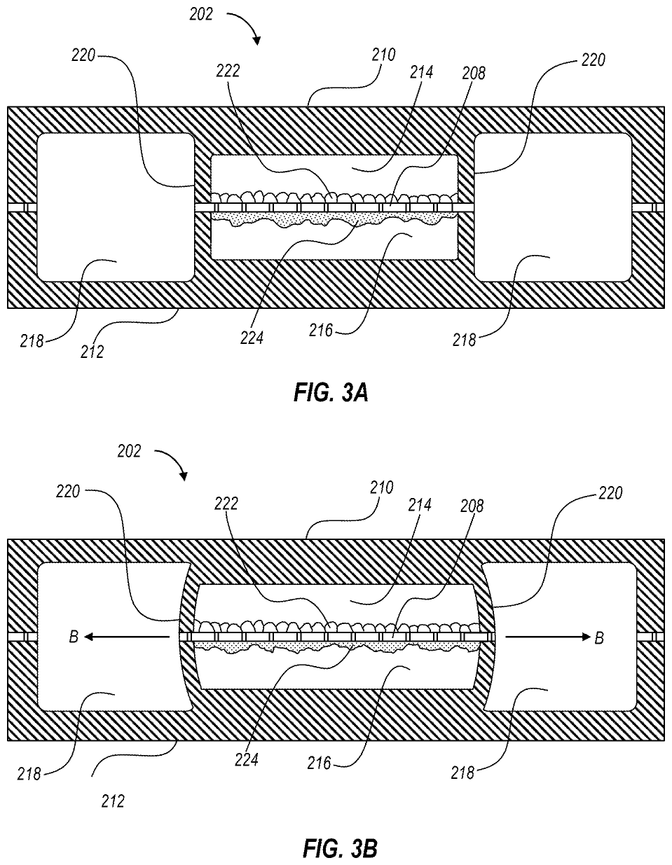

FIGS. 3A and 3B illustrate a cross-sectional view of the cell component 202 of the MPS 200 of FIG. 2 along line A, with FIG. 3A presenting the cell component 202 in a relaxed configuration and FIG. 3B presenting the cell component 202 in a “stretched” configuration. The cell component 202 comprises a culturing membrane 208 that is placed between an upper body portion 210 and a lower body portion 212. The body portions 210 and 212 are typically formed from PDMS using soft lithography, enabling the system 200 to be produced using a mold and rendering the system 200 transparent so that the surface of the membrane 208 may be imaged.

The cell component 202 comprises three microfluidic channels 206: a cell channel, sub-divided further by the culturing membrane 208 to form an air sub-channel 214; and a cell medium sub-channel 216, and two vacuum channels 218, disposed to either side of the cell channel. The cell channel is separated from the vacuum channels 218 by two interior walls 220. The walls of the vacuum channels 218 are typically coated with an acidic compound so as to etch the culturing membrane 208 from within the vacuum channels 218.

The culturing membrane 208 of the exemplary MPS 200 is typically formed from elastomeric materials, preferably PDMS. The membrane 208 may be permeable or semi-permeable to allow the transport of material from the cell medium sub-channel 216 to the surface of the air sub-channel 214. Epithelial cells 222 are disposed on the top surface of the membrane while endothelial cells 224 are disposed on the bottom surface of the membrane. Air may be provided through the microfluidic channels 206 of the system 200 to the air sub-channel 214, while cell media may be provided through the microfluidic channels 206 to the cell medium sub-channel 216.

The membrane 208 may be stretched so as to model the flexing of the alveolar-capillary barrier incident to respiration. FIG. 3A illustrates the cell component 202 in an unstretched position, modeling the alveolar-capillary barrier in a relaxed state when excess air has been expelled from the lung.

The cell component 202 may then be placed in a stretched position by applying a vacuum to the vacuum channels 218 via the microfluidic channels 206 of the system 200, resulting in a pressure differential between the vacuum channels 218 and the cell channel. The pressure differential causes the inner walls 220 of the cell component 202 to flex outward. Because the culturing membrane 208 is attached to the inner walls 220, it is stretched along axis B. By alternating the state of the cell component 202 from a relaxed state to a stretched state back to the relaxed state, the system 200 can expose the membrane 208, and the epithelial 222 and endothelial 224 cells deposited thereon, to uniaxial cyclical strain.

However, the above described stretching of the membrane 208 exposes the cells therein only to uniaxial strain, as the membrane 208 is only stretched along one axis (i.e., axis B) of the system 200. The cyclical strain described above does not subject the cells 222 and 224 to radial strain that exists in-vivo at the alveolar-capillary barrier of the lungs as a result of the semi-spherical shape of the alveoli.

The MPS 200, and similar devices, present other limitations. Systems such as system 200 have traditionally been produced using soft lithography techniques to produce the microfluidic channels 206. PDMS is typically employed to create the cell component 202 (including the culturing membrane 208) because of its material benefits and manufacturing properties, including permeability to gases such as oxygen and carbon dioxide, relative ease of fabrication, optical transparency, elastomeric properties, cell compatibility, and manufacturing precision within the micrometer range. However, PDMS suffers from significant drawbacks, including absorption of small hydrophobic molecules (e.g., pharmaceuticals introduced to the system for study, components of the cell media, etc.) that may complicate characterization of the system and confinement to planar layers of construction, limiting the possible three-dimensional architectures available.

Moreover, soft lithography includes many fabrication steps, including application of a photoresist and a mask on a silicon wafer for generating the PDMS mold, typically in a clean room, which can increase the cost and complexity of fabricating the system. The fabrication steps are generally also performed manually, increasing the time and potential for error (as each successive layer needs to be properly aligned, cored, and plasma bonded) as well as requiring the skills of a trained technician. Furthermore, as a synthetic polymer, PDMS doesn't replicate the complex microenvironment necessary for more physiologically relevant cell behavior.

### Example Improved Microphysiological Systems

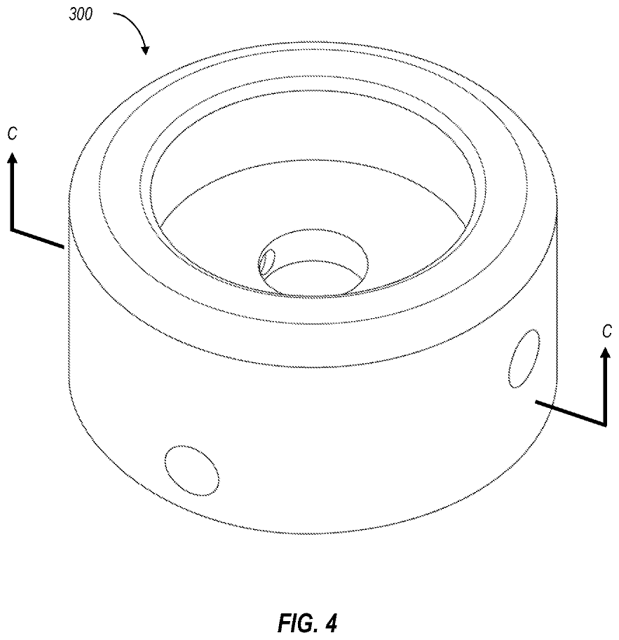

FIG. 4 illustrates an example embodiment of an improved lung MPS 300. The system 300 can better model the microenvironment of the distal lung by introducing cyclic radial strain to a culturing membrane. The system 300 may be relatively small and may have a width within a range of approximately 5 mm to approximately 50 mm.

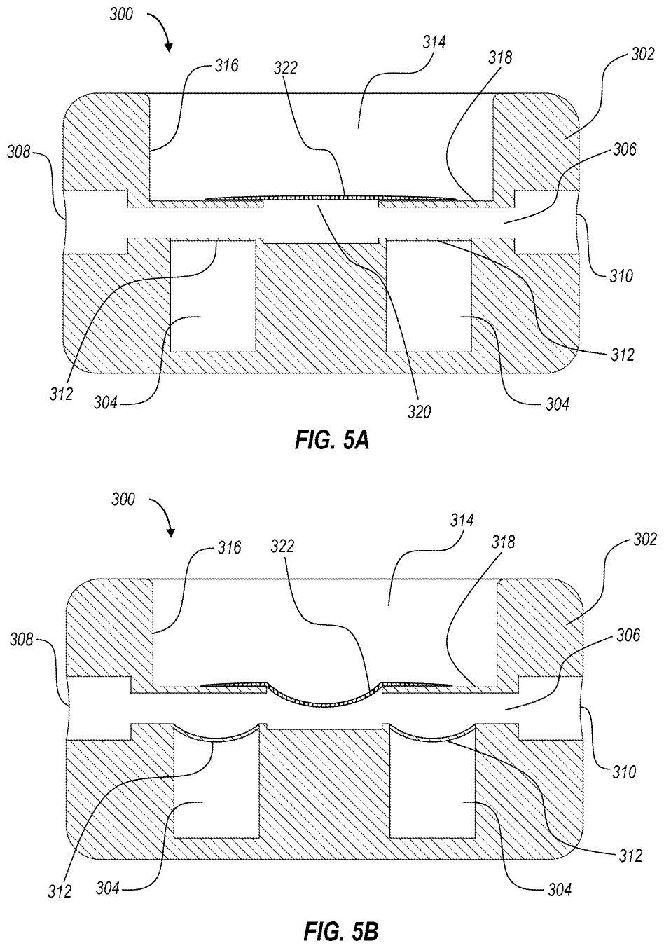

FIGS. 5A and 5B illustrate front cross-sectional views of the MPS 300, showing the interior of the system 300. The system 300 includes a body 302 having multiple microfluidic features. The first microfluidic feature comprises one or more vacuum channels 304 extending from a vacuum inlet into the body 302 to a terminal portion. The illustrated embodiment includes two vacuum channels 304, though other embodiments may include a single vacuum channel 304 or more than two vacuum channels. The system 300 also includes a second microfluidic feature, referred to herein as a circulation channel 306, including a circulation inlet 308 and a circulation outlet 310.

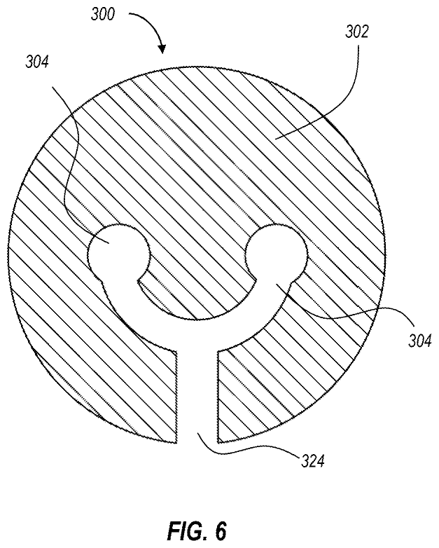

FIG. 6 illustrates a top cross-sectional view of the system 300 along line C of FIG. 4. Illustrated is the vacuum inlet 324 that then branches into two vacuum channels 304 on either side of the system 300, such that the vacuum channels 304 are positioned laterally and below, but not positioned directly beneath, the aperture 320. While the one or more vacuum channels 304 of the system 300 may be positioned directly beneath the aperture 320, it is preferable that the one or more vacuum channels 304 are positioned laterally of an axis running through the center of the aperture 320 so as not to interfere with imaging of the culturing membrane 322. The one or more vacuum channels 304 may be oriented below, above, or lateral to the circulation channel 306, although preferably the one or more vacuum channels 304 are located below the circulation channel 306 so as to reduce the width of the system 300.

As best shown in FIGS. 5A and 5B, the circulation channel 306 is separated from each of the one or more vacuum channels 304 by an actuation diaphragm 312, which comprises a flexible barrier. As illustrated, each vacuum channel 304 may be associated with a corresponding actuation diaphragm 312.

Each actuation diaphragm 312 may be sufficiently thin so as to allow the diaphragm 312 to be sufficiently flexible and impart a pressure differential across the diaphragm 312. The actuation diaphragm 312 may have a thickness within a range of approximately 15 μm to approximately 95 μm, or approximately 25 μm to approximately 85 μm, or approximately 35 μm to approximately 75 μm, or approximately 45 μm to approximately 65 μm, or approximately 45 μm to approximately 55 μm, or approximately 50 μm, or a range with endpoints having any two of the foregoing values. The actuation diaphragm 312 may be formed from a single layer of printed resin or photopolymer, such that the actuation diaphragm 312 has a thickness similar to the resolution of the 3D printing technology employed.

The microfluidic features, or portions thereof, may have a diameter of approximately 50 μm to approximately 5 mm, or approximately 100 μm to approximately 1 mm, or approximately 500 μm, or a range with any two of the foregoing values as endpoints.

A cavity 314 may be formed at or near the top of the body 302, defined by a cavity wall 316 and a rim 318. An aperture 320 may also be formed in or near the center of the rim 318. The aperture 320 of the rim 318 is preferably circular in shape, but may comprise other shapes, such as a triangular, square, or other polygonal shape, or may have an irregular shape.

### Example Culturing Membrane

A culturing membrane 322 may be placed or otherwise disposed over the rim 318 and the aperture 320. In some embodiments, the culturing membrane 322 may omit adhesive or other structure for affixing the culturing membrane 322 to the rim 318. Beneficially, the system enables weak forces, such as hydrogen bonding, van der Waals forces, and surface tension to hold the membrane 322 in place, including during flexion of the membrane 322.

The culturing membrane 322 may comprise various natural or synthetic polymers. Preferably, the culturing membrane 322 is composed of materials that enable the culturing membrane to undergo and withstand cyclic actuation, have a membrane thickness similar to that of the alveolar-capillary barrier (200 nm to 2 μm), and/or have sufficient optical transparency for viewing under a standard light microscope. The membrane 322 may have sufficient permeability to nutrients and signaling molecules and also permit the transmigration of immune cells and fibroblasts.

The culturing membrane 322 can be composed of proteins included in the human matrisome, such that the culturing membrane comprises an extracellular matrix (ECM) membrane. The culturing membrane 322 may comprise collagen (e.g., types I and III) and elastin so as to replicate the primary constituents of the human lung matrisome. The culturing membrane 322 may additionally or alternatively include other proteins of the matrisome, including laminin, type IV collagen, type V collagen, entactin, chondroitin sulfate, chondroitin sulfate proteoglycan, heparan sulfate proteoglycan, and combinations thereof.

Preferably, the culturing membrane 322 includes at least one type of collagen and at least one type of elastin. The culturing membrane 322 may contain substantially equal parts collagen and elastin. ECM membranes primarily composed of collagen and elastin were found to better approximate the general architecture of the lung ECM and better impart pulmonary compliance and elastic recoil.

For example, an extracellular membrane may be formed by a solution of 4 mg/mL of rat tail collagen (e.g., RatCol® Advanced Biomatrix in 0.02M ascetic acid) and 4 mg of solubilized elastin powder (e.g., bovine elastin solubilized via partial hydrolysis in oxalic acid) mixed at a concentration of 4 mg/mL, and then neutralizing the solution with 1M NaOH to form an ECM gel. The gel can then be disposed upon the rim 318 of the system 300 and allowed to dry.

It is preferable that the aperture 320 has a sufficiently small diameter such that the culturing membrane 322 does not fall through the aperture 320. For example, the aperture 320 may have a diameter of approximately 0.5 mm to approximately 10 mm, or approximately 1 mm to approximately 5 mm, or approximately 2.5 mm, or be within a range that uses any two of the foregoing values as endpoints. A thin film comprising the culturing membrane 322 is formed after drying of the ECM gel, upon which cells may then be cultured.

The MPS will more closely approximate the lung microenvironment as the thickness of the ECM membrane approaches the thickness of the alveolar-capillary barrier (i.e., approximately 200 nm to 2 μm), although characteristics of the ECM gel may limit the formation of a culturing membrane 322 with minimal thickness. After drying, the culturing membrane 322 may have a thickness within a range of approximately 0.5 μm to approximately 100 μm, approximately 1 μm to approximately 50 μm, approximately 2.5 μm to approximately 20 μm, approximately 5 μm to approximately 10 μm, or a range with endpoints using any two of the foregoing values.

Epithelial cells (e.g., NCI-H441 cells) may be disposed on the top surface of the culturing membrane 322 and endothelial cells (e.g., human microvascular endothelial cells (HMVEC), human lung microvasculature cell line (HULEC-5a), and/or human umbilical vein endothelial cells (HUVEC)) may be disposed on the bottom surface of the culturing membrane 322, such that the MPS 300 models the microenvironment of the distal lung.

For example, the MPS 300 may first be inverted and a cell media containing the desired endothelial cells may be introduced into the circulation channel 306, allowing the endothelial cells to settle onto a bottom surface of the culturing membrane 322. After the endothelial cells have adhered to the bottom surface of the membrane 322 (e.g., twenty-four hours), the MPS 300 may be inverted again and the top surface of the culturing membrane 322 seeded with epithelial cells and covered in cell media. After the epithelial cells have reached full confluency the cell supernatant may be removed, creating an air-liquid interface. Introduction of mitomycin C or UV treatment may be used to inhibit over-proliferation of the epithelial and endothelial cells.

### Example System Operation

An incompressible medium may fill or may otherwise flow within the circulation channel 306 so as to maintain the air-liquid interface with the cells of the culturing membrane 322. Preferably, a cell media may continuously perfuse through the circulation channel 306 so as to provide the endothelial cells with needed nutrients and shear forces to the culturing membrane 322 and cells deposited thereon, and may mimic the flow of blood through the capillaries of the lung. The cell medium may flow from a reservoir through the circulation inlet 308, the circulation channel 306, and then out the circulation outlet 310.

A syringe pump (or other pump), connected to the circulation inlet 308 or circulation outlet 310 of the system 300 may be used to deliver the required flow of incompressible medium. It is preferable that the pump be capable of accurately controlling the flow of the incompressible medium, especially in instances when a pressure differential (e.g., at least −4 psi) exists across the actuation diaphragm 312. Thus, deflection of the actuation diaphragm 312 by a vacuum causes a similar deflection in the culturing membrane 322 and does not alter the flow of the incompressible medium.

The flow rate of the incompressible medium or fluid may be relatively small, such as approximately 1 μL/hour to approximately 25 μL/hour, or approximately 5 μL/hour to approximately 20 μL/hour, or approximately 10 μL/hour to approximately 15 μL/hour, approximately 12.5 μL/hour, or be within a range using any two of the foregoing values as endpoints.

FIG. 5A illustrates the system wherein the actuation diaphragm 312 and the culturing membrane 322 are in a relaxed state. In this state, the one or more vacuum channels 304 are subjected to a pressure at or near the ambient pressure of the system 300, such that the actuation diaphragm 312 is not deflected.

In contrast, FIG. 5B illustrates the system 300 in a flexed state wherein the one or more vacuum channels 304 are subjected to a vacuum. The vacuum of the vacuum channels 304 creates a pressure differential between the vacuum channels 304 and the circulation channel 306, causing the actuation diaphragm 312 to deflect. The incompressibility of the fluid transmits the pressure differential to the culturing membrane 322, causing the culturing membrane 322 to deflect as well, with the deflection of the culturing membrane 322 typically being greatest at the center and decreasing radially therefrom. Strain is imparted to the cells as the culturing membrane 322 is deflected.

The one or more vacuum channels 304 may be exposed to a vacuum sufficient to induce an approximately 4% to approximately 12% strain in the culturing membrane 322. The strain may be measured as the difference between the length of the relevant portion of the membrane 322 (i.e., the portion of the membrane 322 disposed over the aperture 320) in a relaxed state and the length of the portion in a deflected state, divided by the length of the portion in a relaxed state). Such strain levels are similar to the strain experienced at the in vivo alveolar-capillary barrier. For example, it has been found that a vacuum of approximately −4 psi is sufficient to induce approximately 10% strain in an ECM membrane positioned on an aperture 320 having a diameter of approximately 2.5 mm. The system 300 may then be returned to a relaxed state by reducing the vacuum from the one or more vacuum channels 304.

By providing cyclic application of vacuum at a rate similar to that of human respiration (e.g., approximately 12 to approximately 20 times per minute) and at strain levels similar to the in vivo environment, the system 300 may provide cyclic radial strain to the cells in a manner similar to that exhibited at the alveolar-capillary barrier.

### Example Manufacturing Methods

The system 300 is preferably formed such that the body 302 and actuation diaphragm 312 are composed of the same material and comprise a contiguous, integrated whole. Various manufacturing methods are known in the art capable of printing the MPS body 302 and actuation diaphragm 312, including 3D printing technology such as stereolithography (SLA), masked stereolithography apparatus (MSLA), and digital light processing (DLP) printing, which are proficient at closing open cavities and have good resolution for forming microfluidic features. Various photopolymers or light-activated resins may be employed. For example, a suitable resin is NextDent Ortho Flex®, a resin having good characteristics such as providing a semi-transparent surface, good flexural strength, and low water sorption.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A lung microphysiological system, comprising:
a body;
a first microfluidic feature comprising one or more vacuum channels extending into the body from a vacuum inlet to a terminal portion within the body; and
a second microfluidic feature comprising a circulation channel extending into the body from a circulation inlet and exiting the body at a circulation outlet,
wherein at least one of the one or more vacuum channels is separated from the circulation channel by a flexible barrier.

2. The system of claim 1, wherein the body and the flexible barrier are integrally formed as a single, contiguous material.

3. The system of claim 1, wherein the vacuum inlet is the only inlet of the first microfluidic feature.

4. The system of claim 1, wherein the first microfluidic feature comprises multiple vacuum channels, each vacuum channel being separated from the circulation channel by a flexible barrier.

5. The system of claim 1, wherein the first microfluidic feature is configured to provide cyclic strain to cells disposed in the circulation channel through cyclic application of vacuum to the vacuum channel, thereby causing cyclic deflection of the flexible barrier which transfers cyclic flexing to the cells.

6. The system of claim 1, wherein the second microfluidic feature is configured to provide flow of an incompressible medium across cells disposed in the circulation channel.

7. The system of claim 6, wherein a flow of an incompressible medium through the circulation channel mimics blood flow.

8. The system of claim 1, wherein the body is a polymer material.

9. The system of claim 1, wherein the body includes an aperture disposed above the circulation channel.

10. The system of claim 9, wherein the one or more vacuum channels are not disposed directly beneath the aperture.

11. The system of claim 9, wherein a culturing membrane is disposed over the aperture.

12. The system of claim 11, wherein the culturing membrane comprises an extracellular matrix (ECM) to form an ECM membrane.

13. The system of claim 12, wherein the ECM membrane comprises collagen and elastin.

14. The system of claim 12, wherein one or more layers of endothelial cells are disposed on a lower side of the ECM membrane and are thereby exposed to the circulation channel.

15. The system of claim 12, wherein one or more layers of epithelial cells are disposed on an upper side of the ECM membrane.

16. The system of claim 12, wherein the ECM membrane is held in position via weak bonding to the body and/or surface tension, without an adhesive.

17. The system of claim 1, wherein a thickness of at least one flexible barrier is within a range of approximately 15 nm to approximately 95 nm.

18. The system of claim 1, wherein the flexible barrier is formed from a single layer of 3D printed polymer.

19. A lung microphysiological system, comprising:
a body;
a first microfluidic feature comprising multiple vacuum channels extending into the body from a vacuum inlet, wherein the vacuum inlet is the only inlet of the first microfluidic feature;
a second microfluidic feature comprising a circulation channel extending into the body from a circulation inlet and exiting the body at a circulation outlet;
a set of flexible barriers, each flexible barrier disposed to separate a respective vacuum channel from the circulation channel; and
an aperture disposed above the circulation channel,
wherein the body and the flexible barriers are integrally formed as a single, contiguous material.

20. The system of claim 19, wherein the one or more vacuum channels are not disposed directly beneath the aperture.
